(19) United States
(12) Patent Application Publication
(10) Pub. No.: US 2022/0195390 A1
(43) Pub. Date: Jun. 23, 2022

(54) METHODS FOR PREPARING EXTRACELLULAR VESICLES (EV) DEPLETEMEDIA

(71) Applicants: INSERM (Institut National de la Santé et de la Recherche Médicale), Paris (FR); UNIVERSITÉ PARIS-SACLAY, GIF-SUR-YVETTE (FR); ETAT FRANÇAIS, SERVICE DE SANTÉ DES ARMÉES REPRÉSENTÉ PAR LE DÉLÉGUÉ GÉNÉRAL DE L'ARMEMENT, Paris Cedex 15 (FR)

(72) Inventors: Georges UZAN, Villejuif (FR); Jean-Jacques LATAILLADE, Villejuif (US); Philippe MAUDUIT, Villejuif (FR); Sébastien BANZET, Villejuif (FR); Sylvie GOULINET, Villejuif (FR); Juliette PELTZER, Villejuif (FR); Bastien RIVAL, Villejuif (FR)

(21) Appl. No.: 17/429,201

(22) PCT Filed: Feb. 6, 2020
(86) PCT No.: PCT/EP2020/053049

§ 371 Date: Aug. 6, 2021

(30) Foreign Application Priority Data
Feb. 7, 2019 (EP) 19305142.2

(51) Int. Cl. C12N 5/078; C12N 5/0775; A61K 35/19; A61K 35/28; A61K 47/69

(52) U.S. Cl. CPC C12N 5/0644; C12N 5/0663; C12N 2500/90; A61K 35/28; A61K 47/6901; A61K 35/19

(57) ABSTRACT

Cell therapy is getting a growing interest in a wide range of indications in human. In many cases, a substantial part of the therapeutic effects relies on cell-secreted factors and the extracellular vesicles (EV) are proposed as a cell-free surrogate for cell therapy. Currently, during the EV production phase, human cells are placed in serum-free media to produce EV, with limited cell survival. Here, the inventors describe a new procedure for GMP-compatible human cells-derived EV wherein Human Platelet Lysate (HPL) is produced from which the EV are removed by tangential-flow-filtration resulting in an EV-depleted HPL. Said EV-depleted HPL may be then uses as a culture medium for the production of EV by cells of interest.

### FIELD OF THE INVENTION

The present invention relates to methods for preparing extracellular vesicles (EV) depleted-media that are suitable for producing EV from a population of cells of interest.

### BACKGROUND OF THE INVENTION

Human cells use multiple and sophisticated modes of communication. These include secretion of cytokines, chemokines or growth factors, direct cellular communication through the expression of different cell surface markers and also production of extracellular vesicles (EV) containing proteins, DNA, mRNA, miRNA . . . . This intercellular communication via extracellular cargo is highly conserved across species (from bacteria to human) and therefore EV are likely to be a highly efficient, robust and economic manner of exchanging information between cells.

EV could protect cells from accumulation of waste or drugs, contribute to physiology and pathology and therefore have a myriad of potential clinical applications, ranging from biomarkers to anticancer therapy. They also could cross the blood-brain barrier. These EV could recapitulate most effects of the cells from which they originate and be used as a substitute to those cells in therapeutic objectives.

EV must be characterized during large-scale production, frozen in a convenient way for being stored and conveyed and being immediately available as a therapeutic agent. Typically, EV can be isolated from the culture media using various methods including ultra-centrifugation, tangential filtration, immuno-capture, precipitation . . . . Culture media commonly used for culturing cells require serum or platelet lysate that contain large amounts of EV that cannot be distinguished and separated from the cell-secreted EV. Purification and characterization of EV from culture cells therefore needs the prior elimination of exogenous EV contained in serum (for example Fetal Bovine Serum, FBS) or Human Platelet Lysate (HPL). An unsatisfactory solution (the most used today) is to incubate the cells in the absence of serum or platelet lysate during the production phase of these EV. However, these culture conditions are stressful and alter the physiology of the cells relative to that observed in their initial culture conditions in HPL or FBS containing medium. Moreover, cells cannot be cultured for a long time under these starving conditions, and this substantially limits the production of EV. Another option would be to use serum- or HPL-free complete culture media (containing specific cocktails of growth factors and additives). However these media have important limitations, they are very cell specific, some cells cannot be grown with these media and they are very expensive, which substantially limits the possibility of producing large volumes of conditioned media. One way to partly overcome these problems would be to use commercially available “exosome-free” serum. However, these preparations are only 90% depleted. The residual presence of such amounts of bovine EV preclude the clinical use of these vesicles. Moreover, regulatory authorities recommend avoiding animal components of the cell expansion process, thus excluding FBS. The potential risk of contamination by FBS leads to prefer the use of xenobiotic-free culture conditions. Consequently, xenobiotic-free culture conditions have also to be considered for EV production. In these conditions, only HPL offers such a possibility. HPL is a useful substitute to FBS to isolate, amplify and maintain human cells. However, up to now, the production and use of EV-depleted HPL to produce highly purified EV of cultured cells has never been reported.

### SUMMARY OF THE INVENTION

As defined by the claims, the present invention relates to methods for preparing extracellular vesicles (EV) depleted-media that are suitable for producing EV from a population of cells of interest.

### DETAILED DESCRIPTION OF THE INVENTION

Cell therapy is getting a growing interest in a wide range of indications in human. In many cases, a substantial part of the therapeutic effects relies on cell-secreted factors and the extracellular vesicles (EV) are proposed as a cell-free surrogate for cell therapy. Currently, during the EV production phase, human cells are placed in serum-free media to produce EV, with limited cell survival. Here, the inventors describe a new procedure for GMP-compatible human cells-derived EV. Human Plasma Lysate (HPL) is produced following the removing of the EV by tangential-flow-filtration resulting in an EV-free HPL. Said EV-free HPL may be then used as a culture medium for the production of EV by cells of interest.

Accordingly the first object of the present invention relates to a method for removing extracellular vesicles from a medium comprising the steps of i) filtering the medium by tangential-flow filtration with a filter having a pore size between 100 kDa and 50 nm and wherein the trans-membrane pressure (TMP) is between 1 and 6 psi and the shear rate is between 2000 and 8000 s⁻¹ and ii) collecting the permeate after said tangential-flow filtration.

As used herein, the term “medium”, as used in reference to a cell culture, includes the components of the environment surrounding the cells. In some embodiments, the medium is a serum free medium. As used herein, the term “serum-free” as used herein, is understood as being devoid of human or animal serum. In some embodiments, the medium is a platelet lysate and more particularly a human platelet lysate. As used herein the term, the term “platelet lysate” refers to the products of platelet lysis. The platelet lysate may also include any medium in which the lysed platelets are contained. Freezing and thawing is the typical, but not the only, method for lysing platelets in this disclosure. Mechanical lysis, typically through the use of shear forces, is another method contemplated for producing a lysate. Lysis buffers, typically acting by placing the cells in a hypotonic solution, are yet another option. The lysis process may consist of combinations of these methods. In some embodiments, the resulting lysate is combined with cell culture media. In some embodiments, the serum-free platelet lysate medium includes PLTMax® platelet lysate. For instance, the PLTMax® platelet lysate may be present in an amount of 5 wt % based on the total weight of the serum-free platelet lysate-containing medium that is free of feeder cells.

As used herein, the term “extracellular vesicle” or “EV” has its general meaning in the art and is a collective term for different types of membrane-surrounded structures with overlapping composition, density, and sizes (ranging from 30 to >1,000 nm in diameter). In accordance with the recommendations of the International Society for Extracellular Vesicles (ISEV), the term includes but is not limited to exosomes, ectosomes, microvesicles particles apoptotic bodies, argosomes, blebbing vesicules, budding vesicules, dexosomes, ectosomes, exosomes-like vesicules, exosomes, exovesicules, extracellular membrane vesicules, matrix vesicules, membrane particules, membrane vesicules, microparticules, microvesicles , nanovesicles, oncosomes, prominosomes, prostasomes, shedding microvesicles, shedding vesicles, and tolerosomes. Up to now EV are considered to be secreted by all cell types and are present in high amount in all biological fluids analyzed so far (serum, urine/plasma, saliva, cerebrospinal fluid . . . ). EV may have a diameter (or largest dimension where the particle is not spheroid) of between about 10 nm to about 5000 nm (e.g., between about 50 nm and 1500 nm, between about 75 nm and 1500 nm, between about 75 nm and 1250 nm, between about 50 nm and 1250 nm, between about 30 nm and 1000 nm, between about 50 nm and 1000 nm, between about 100 nm and 1000 nm, between about 50 nm and 750 nm, etc.).

As used herein, the term “removing” may refer to complete removal of the EV in the medium after the filtering step.

As used herein, the term “tangential-flow filtration” or “TFF” refers to a process in which the medium containing the EV to be removed by filtration is circulated at high velocities tangential to the plane of a filter membrane. In such filtrations a pressure differential is applied along the length of the membrane to cause the fluid and filterable solutes to flow through the filter. According to the invention, the filter used with the invention will be selected such that all of EV remains in the retentate, whereas the other components of the medium pass into the permeate that may be re-circulated to the feed reservoir to be re-filtered in additional cycles. As used hereinafter, the term “retentate” refers to the materials that flow by the surface of the filter in a TFF device but do not pass through the filter. When a fluid composition flow through a TFF device, particles (e.g. EV) with sizes larger than the average pore size of the TFF filter cannot pass through the filter and are likely to remain as the components of the retentate. As used hereinafter, the term “permeate” refers to the materials that pass through the filter in the TFF device. When a fluid composition flow through a TFF device, particles with sizes smaller than the average pore size of the TFF filter may pass through the filter and become components of the permeate.

The filter membrane has a pore size that is large enough to allow the medium to pass through and small enough to retain EV. The filter that is used for the TFF is thus characterized by a pore size. As used herein, the term “pore size” refers to the average size of the smallest particle that a stationary phase will reject or that a membrane will retain on the sample side. The size is typically expressed in particle diameter or molecular mass. Membrane pore size is usually stated in kDa and refers to the average molecular mass of the smallest particle or macromolecule the membrane is likely to retain. Alternatively, membrane pore size can be stated in nanometer (nm) and refers to the diameter of the smallest particle the membrane is likely to retain. The diameter is proportional to the molecular mass for molecules of a similar shape (e.g. spherical molecules). The inventors have found that a pore size between 100 kDa and 50nm is preferred. For instance, a pore size of about 100 kDa, about 150 kDa, about 200 kDa, about 250 kDa, about, 300 kDa, about 350 kDa, about 400 kDa, about 500 kDa, about 550 kDa, about 600 kDa, about 650 kDa, about 700, kDa, about 750 kDa, about 750 kDa, about 800 kDa, about 850 kDa, about 900 kDa, about 950 kDa, about 1000 kDa may be used. Ideally, pore size is set to about 500 kDa. The filter of the present invention typically comprises a number of pores distributed across the area of the filter. In some embodiments, the filter has a pore size with a small variation in pore size. For example, the variability in the pore size can be about ±20%, or within the range of about +0 to about 20%.

The filter membrane can be made of any suitable material. Such filters include, but are not limited to, microporous membranes of nylon, polyvinylidene fluoride (PVDF), cellulose acetate/nitrate, polysulfone, modified polyethersulfone, polycarbonate, polyethylene, polyester, polypropylene, and polyamide. Other filters, such as ceramic filters and metallic filters, can also be used. Both hydrophilic and hydrophobic, charged and uncharged filters can be used. In some embodiments, hydrophilic filters can be preferred. In some embodiments, the filter comprises a hollow fiber module comprising a bundle of filter membranes, each filter membrane being shaped in the form of a hollow tube. In this case, the feed stream is pumped into the lumen of the tubes such that permeate passes through the membrane to the shell side, where it is removed. Typically, the hollow tube comprises a diameter between about 0.1 to about 2.0 mm. In some embodiments, the filter membrane has an inner diameter of at least 0.5 mm. In some embodiments, the filter comprises a flat plate (or cassette or capsule) module comprising layers of membrane, with or without alternating layers of separator screen, stacked together and sealed in a package. Feed fluid is pumped into alternating channels at one end of the stack and the permeate passes through the membrane into the permeate channels. Filter membranes may vary according to their effective surface area. The effective membrane surface area is typically stated in cm² and refers to the total surface of the filter membrane that is exposed to the medium. The effective surface area for hollow fibre membranes depends on the average diameter and effective length of the fibres and the total number of fibres.

According to the present invention a predetermined transmembrane pressure is applied during the process. As used herein, the term “transmembrane pressure” or “TMP” refers to the pressure differential gradient that is applied along the length of a filtration membrane to cause fluid and filterable solutes to flow through the filter. The unit of measurement for TMP is pound per square inch or psi. The formula of TMP is TMP=Pf−Pp wherein Pf represents the pressure on the feedwater side of the membrane and Pp represents the pressure on the filtrate side of the membrane. Ideally, the transmembrane pressure is chosen so that a high flux of the fluid across the membrane is achieved while maintaining efficient removal of the EV. The inventors have found that a TMP between 1 psi and 6 psi is preferred. For instance, a TMP of about 1 psi, about 1.5 psi, about 2 psi, about 2.5 psi, about 3 psi, about 3.5 psi, about 4 psi, about 4.5 psi, about 5 psi, about 5.5 psi or about 6 psi may be used. Ideally, the transmembrane pressure is set to about 2 psi (13790 Pa).

According to the present invention a predetermined shear rate is applied during the process. As used herein, the term “shear rate” refers to the parameter used to characterize laminar flow. The SI unit of measurement for shear rate is s⁻¹, expressed as “reciprocal seconds” or “inverse seconds.” The formula of the shear rate (γ) is: γ=f(k) 6 Q/ab² wherein γ is the shear rate (in s⁻¹), Q is the flow rate (ml/s), a=slit width (cm), b=slit height (cm) and f(k) is a function of the physical parameters of the system. Ideally, the shear rate is chosen so that a high flux of the fluid through the filter is achieved while maintaining EV integrity and avoiding the formation of a gel layer on the surface of the filter membrane. Typically, the shear rate may be adjusted by controlling the flow rate. The inventors have found that a shear rate between 2000 and 8000 s⁻¹ is preferred. For example, a shear rate of about 2000 s⁻¹, about 2500 s⁻¹, about 3000 s⁻¹, about 3500 s⁻¹, about 4000 s⁻¹, about 4500 s⁻¹, about 5000 s⁻¹, about 5500 s⁻¹, about 6000 s⁻¹, about 6500 s⁻¹ about 7000 s⁻¹, about 7500 s⁻¹, or about 8000 s⁻¹ may be used. Ideally, the shear rate is set to about 4000 s⁻¹.

As used herein, the term “about,” as applied to one or more values of interest, refers to a value that is similar to a stated reference value. In some embodiments, the term “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction of the stated reference value unless otherwise stated or otherwise evident from the context.

Many TFF systems are commercially available (e.g. using hollow fibres such as those available from GE Healthcare and Spectrum Labs). Typically, the devices comprise a cross-flow chamber and a filtrate chamber. The filter is positioned between and with one surface in fluid communication with the cross-flow chamber (the retentate surface) and other surface in fluid communication with the filtrate chamber (the permeate surface). The cross-flow chamber, filtrate chamber and filter comprise a remover unit. The medium enters the cross-flow chamber through a fluid inlet that is typically situated adjacent to the retentate surface of the filter and such that the medium enters the chamber substantially parallel to the retentate surface of the filter. Typically, fluid is removed from the cross-flow chamber through a fluid outlet, which is usually located at a portion of a cross-flow chamber perpendicular to the retentate surface of the filter. In some embodiments, the medium is passed across the retentate surface of the filter by pumping the medium into the cross-flow chamber. The pump used to drive the cross-flow of fluid across the filter is referred to as the “cross-flow pump” or “recirculating pump”. The cross-flow pump can include any pumping device in fluid communication with the cross-flow chamber sufficient to introduce the flow of fluid into the chamber and across the filter at the specified input rate, without causing substantial damage to EV. A cross-flow pump suitable for use in the present invention can include, e.g., a peristaltic pump, piston pump, diaphragm pump, or roller pump. A peristaltic pump can be used, for example, where it is desired to maintain the TFF device as part of a “closed” system.

The EV-depleted medium may find various applications. In particular, said EV-depleted medium can be used for the production of EV from a cell-type of interest and thus allowing that the produced EV are not contaminated by some EV already present in the culture medium.

Thus a further object of the present invention relates to a method for producing EV from a population of cells comprising the steps of i) preparing an EV-deplete medium by the method as disclosed herein, ii) culturing the population of cells in a culture medium supplemented by the EV-depleted medium as prepared a step i) in condition for allowing the production of EV by the cells and iii) harvesting the EV that are produced at step ii).

As used herein, the term “cell” refers to any eukaryotic cell. Eukaryotic cells include without limitation ovary cells, epithelial cells, circulating immune cells, hematopoietic cells, bone marrow cells, circulating vascular progenitor cells, cardiac cells, chondrocytes, bone cells, beta cells, hepatocytes, and neurons . . . . Moreover the term includes pluripotent stem cells. As intended herein, the expression “pluripotent stem cells” relates to division-competent cells, which are liable to differentiate in one or more cell types. Preferably, the pluripotent stem cells are not differentiated. Pluripotent stem cells encompass stem cells, in particular adult stem cells (e.g. mesenchymal stem cells (MSC)) and embryonic stem cells. The term also encompasses induced pluripotent stem cells (IPS). Accordingly the term includes purified primary cells and immortalized cell lines. The term also refers to cells in suspension (e.g. circulating leukocytes (PBMC)), or adherent cells (e.g. endothelial cells).

In some embodiments, the cells are in suspension. In some embodiments, a shear stress is applied to said cell suspension.

Alternatively the cells may consist in adherent cells. For example, said cells adhere to a cell culture surface. The term “cell culture surface” or “cell culture matrix” refers to every type of surface or matrix suitable for cell culture. The term “cell culture surface” includes but is not limited to tissue culture plate, dish, well or bottle and any culture support used in bioreactors such as rollerbottles, plate stacks, particulated supports (including microcarriers), fibers or membranes. In a particular embodiment, the culture surface is plastic surface of the culture plate, dish, well or bottle. The cell culture surface is to be compatible with the adhesion of cells.

Any Cell culture medium known in the art may be suitable and typically the culture medium comprises an amount of calcium (Ca2+). Typically the concentration of calcium corresponds to the in vivo physiological concentration of extracellular calcium.

Any method well known in the art may be used for harvesting the produced EV that typically involves ultracentrifugation, ultrafiltration, size exclusion chromatography, precipitation . . . . In some embodiments, EV may be concentrated by ultracentrifugation that is suitable for differential separation of EV from parent cells. In a particular embodiment, the method further comprises a step consisting of isolating the EV of interest from the supernatant of the cells. Standard methods for isolating EV are well known in the art. For example the methods may consist in collecting the population of EV present in the supernatant of the cells and using differential binding partners directed against the specific surface markers of the EV of interest, wherein EV are bound by said binding partners to said surface markers. In some embodiments, fluorescence activated cell sorting (FACS) may be used to separate in the supernatant the desired EV. In some embodiments, magnetic beads may be used to isolate EV (MACS).

In some embodiments, the EV are loaded with an agent, such as a small molecule, a protein or a nucleic acid molecule of interest. Methods for loading EV with agent are known in the art and include lipofection, electroporation, as well as any standard transfection method. In some embodiments, the EV comprising a polynucleotide or polypeptide or small molecule of interest are obtained by over-expressing the polynucleotide or polypeptide or loading the cells with the small molecule in culture and subsequently isolating indirectly modified EV from the cultured cells. In some embodiments, EV comprising a polynucleotide or polypeptide or small molecule of interest are generated by loading previously purified EV with the molecule(s) of interest into/onto the EV by electroporation (polynucleotide or polypeptide), covalent or non-covalent coupling to the EV surface (polynucleotide or polypeptide or small molecule) or simple co-incubation (polynucleotide or polypeptide or small molecule).

The EV are typically prepared as a substantially pure homogenous population of EV obtainable by the method of the invention. The term “substantially pure homogenous population”, as used herein, refers to a population of cell EV wherein the majority (e.g., at least about 80%, preferably at least about 90%, more preferably at least about 95%) of the total number of said cell EV have the specified characteristics of the EV of interest.

The population of cell EV according to the invention may be easily conserved in appropriate medium and therefore may be stored so as to form bank of cell EV.

In some embodiments, the EV prepared by the method as disclosed herein are particularly suitable for use in therapy. Conventional pharmaceutical practice may be employed to provide suitable formulations or compositions to administer the compounds to patients suffering from a disease or disorder. Administration may begin before the patient is symptomatic. Any appropriate route of administration may be employed, for example, administration may be topical, parenteral, intravenous, intraarterial, cutaneous, subcutaneous, intratumoral, intramuscular, intracranial, intraorbital, ophthalmic, intraventricular, intrahepatic, intracapsular, intrathecal, intracisternal, intraperitoneal, intranasal, aerosol, suppository, or oral administration. For example, therapeutic formulations may be in the form of liquid solutions or suspensions; for oral administration, formulations may be in the form of tablets or capsules; and for intranasal formulations, in the form of powders, nasal drops, or aerosols. For example cutaneous administration may be in the form of dressing or cream.

The EV are typically administered to human patients in therapeutically effective amounts (e.g., amounts which prevent, eliminate, or reduce a pathological condition) to provide therapy for a disease or condition. The preferred dosage of an EV of the invention is likely to depend on such variables as the type and extent of the disorder, the overall health status of the particular patient, the formulation of the compound excipients, and its route of administration.

Accordingly, in some embodiments, the EV of the invention may be then mixed with a pharmaceutically-acceptable diluent, carrier, or excipient, to form a pharmaceutical composition that can be administered to a patient suffering from a disease or disorder. As used herein, the term “pharmaceutically acceptable carrier or excipient” refers to a carrier medium which does not interfere with the effectiveness of the biological activity of the cell EV of the invention, and which is not excessively toxic to the host at the concentrations at which it is administered. The term “carrier” refers to a diluent, adjuvant, excipient, or vehicle with which the cell EV of the invention are administered. The composition, if desired, can also contain minor amounts of wetting or emulsifying agents, or pH buffering agents. These compositions can take the form of solutions, suspensions, emulsion, tablets, pills, capsules, powders, sustained-release formulations, dressing, creams, ointments and the like. Examples of suitable pharmaceutical carriers are described in “Remington's Pharmaceutical Sciences” by E.W. Martin. Such compositions will contain a therapeutically effective amount of the population of said EV, preferably in purified form, together with a suitable amount of carrier so as to provide the form for proper administration to the patient. The formulation should suit the mode of administration.

The invention will be further illustrated by the following figures and examples. However, these examples and figures should not be interpreted in any way as limiting the scope of the present invention.

### FIGURES

FIG. 1: Permeate flux expressed as liter/square meter/hour (LMH) as a function of the trans-membrane pressure (psi) following HPL filtration through a 500 kDa pore size hollow fiber filter.

FIG. 2: Permeate flux expressed as liter/square meter/hour (LMH) as a function of the concentration factor of the retentate compartment following filtration of HPL through 100 kDa (triangle), 500 kDa (diamond) or 50 nm (square) pore size hollow fiber filter.

FIG. 3: Size exclusion chromatography analysis of the protein content (OD280) of HPL (HPL mix 41), 100 kDa, 500 kDa and 50 nm HPL permeate.

FIG. 4: Size exclusion chromatography analysis of the protein content (OD280) of 100 kDa, 500 kDa and 50 nm HPL retentate.

FIG. 5: Nanoparticule Tracking Analysis (NTA) quantification of EV removal from HPL by Tangential Flow Filtration. Intensity versus size representation of the population of EV. Taking into account the dilution of the samples and the presence of EV in the diluent (PBS), the depletion obtained in this case is 99.8%.

FIG. 6: Number of cells obtained over 3 cycles of EV production of 72 h. Three conditions are compared: Medium, Medium+EV-Free HPL at 5% and Medium+EV-Free HPL at 8%. The results are presented as ratios. The numbers of cells in the EV-Free HPL conditions are expressed relative to the number in the Medium condition at the same time-point.

FIG. 7: NTA quantification of conditioned medium from MSC incubated for 72 h in α-MEM supplemented with 5% EV-free HPL. Intensity versus size representation of the population of EV produced by MSC in EV-free HPL containing medium.

FIG. 8: EV concentration in conditioned media obtained over 3 cycles of EV production of 72 h. Three conditions are compared: Medium, Medium+EV-Free HPL at 5% and Medium+EV-Free HPL at 8%.

### EXAMPLE 1

We propose a new protocol to produce EV-depleted media from Human Platelet Lysate (HPL). For this, we decided to adopt the tangential flow filtration (TFF) system proposed by

Spectrum Laboratories. The R & D version of this device (KrosFlo Research IIi) combined with a hollow fiber filtration system allows ultrafiltration (molecular weight from 1 to 1000 kDa) and/or microfiltration (Sizes from 0.05 to 0.65 microns) of sample volumes ranging from 1 ml to 10 L. Control of continuous feed rate allow the filtration process to operate at constant shear rate. Filtration is fully controlled by three pressure sensors (feed, retentate and permeate sensor). Associated with an automatic backpressure valve, they allow the control of the trans-membrane pressure (TMP) thus ensuring maximum reproducibility of the process. The range of filters proposed allows the separation and the concentration of soluble biomolecules (mainly proteins) contained in different biological fluids (serum, urine, cerebrospinal fluid . . . ) and media conditioned by cultured cells.

This system, when operated with Spectrum Labs disposable Module-Bag-Tubing (MBT) sets, which are fully assembled and disposable process flow path for TFF, is compatible with regulatory requirements of clinical-grade production units. This gamma-sterile MBT sets are designed for aseptic processing of solution to downstream TFF ultrafiltration. The disposable flow path including the filter, pressure transducers, tubing and fittings completely eliminates the possibility of cross contamination. Last but not least, this process is fully scalable allowing to use hollow-fiber with filtration surface that fit the volume of HPL to be EV-depleted. This allows a cost reduction of research laboratory production.

Using optimized ultrafiltration parameters (filter surface, shear force, TMP and membrane pore size (or MW Cut-off)), we found this system suitable to separate the vesicular component of HPL (vesicles larger than 30 nm) from the soluble proteins part.

Shear force: This force, provided by the circulation rate and applied tangentially to the filtration membrane permanently sweeps any un-filtrated material from the membrane surface thus preventing clogging. It is calculated as: (8×Velocity (m·s⁻¹))/Fiber Internal Diameter (m) with sec−1 units.

A circulation rate (feed rate) that provides an intermediate shear force, between 4,000 and 8,000 sec⁻¹, is a good starting point for processing low fouling streams. However, for feed streams containing fragile components such as extracellular vesicles that may be damaged by high circulation rates or high temperatures, shear forces between 2,000 to 4,000 sec⁻¹ are recommended, therefore 4000 sec⁻¹ was preferred in this study.

Trans Membrane Pressure (TMP): Using pressure transducer, the measurement of feeding pressure (P_{feed}), retentate pressure (Pᵣₑₜₑₙₜₐₜₑ) and permeate pressure (Pₚₑᵣₘₑₐₜₑ) allow the continuous measurement of the trans-membrane pressure (TMP) using the following equation: TMP=((P_{feed}+Pᵣₑₜₑₙₜₐₜₑ)/2)−Pₚₑᵣₘₑₐₜₑ. The use of a backpressure valve that pinch the retentate tubing allows TMP to be automatically and permanently adjusted to a preset value. Processed liquid flux (permeate flux), normalized to the filter surface and expressed as L/m²/h (LMH) will typically increase as a function of TMP. However, depending on the circulation rate, the flux improvement may become asymptotic as TMP increase because of compaction of the macromolecules that create a “gel layer resistance”.

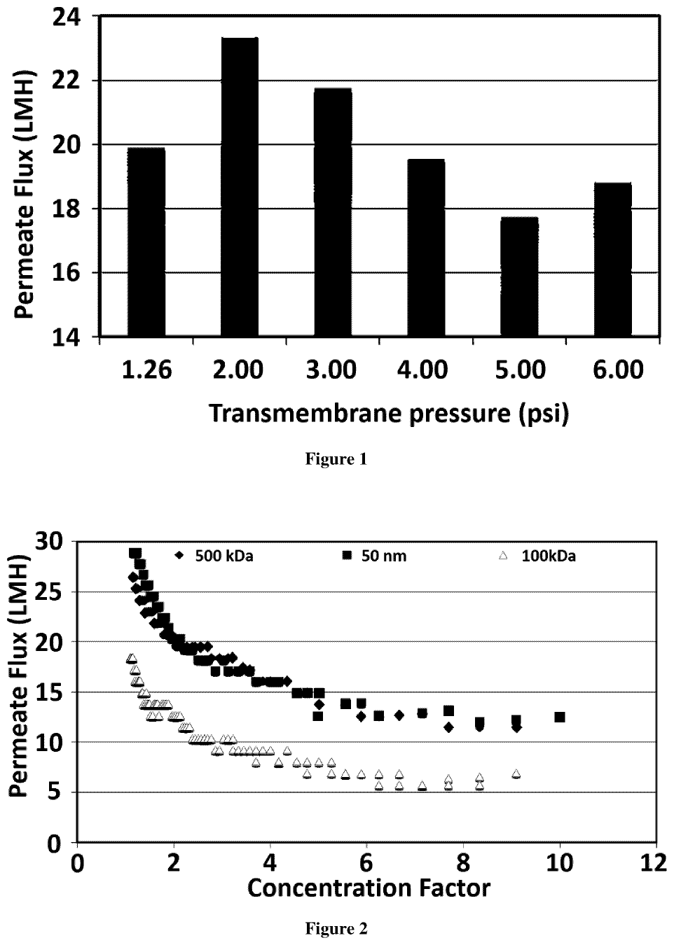

TMP between 1 and 6 psi were tested on the initial filtration phase of HPL (up to 2 fold concentrations of the retentate compartment) through a 500 kDa pore size hollow fiber filter operating at a shear force of 4000 sec−1 (FIG. 1).

All TMP tested can be used in these conditions but a TMP of 2 psi was preferred because it gives the highest permeate flux. This confirms that when filtrating complex solutions such as HPL or serum, limited TMP should be used to favor filtration efficiency. Thus, controlling the permeate backpressure (or permeate flux rate) may reduce the tendency of the membrane to foul in the initial steps of the concentration, providing an overall higher average flux rate.

Membrane pore size: Then, the capacity to deliver EV-free HPL was evaluated using hollow fiber filters of 3 different pore size (100 kDa, 500 kDa and 50 nm). This test was performed with shear force of 4000 s⁻¹ and TMP of 2 psi. FIG. 2 shows the permeate flux as a function of the concentration factor (CF) of the retentate up to 10. This CF allows the production of a filtrated HPL volume of 90% of the initial HPL. In any case, permeate flux decreases rapidly in the initial filtration phase of TFF. Evolution of permeate flux of 500 kDa and 50 nm filters are very similar with values of 19.75 and 20.49 L/m²/h on the overall filtration process. Filtration through 100 kDa filter was nearly 50% less efficient with a mean LMH of 11.53.

TFF through both 500 kDa and 50 nm pore size hollow fiber filters were more efficient than 100 kDa in term of filtration rate.

HPL samples and their different TFF permeate were further analyzed by size exclusion chromatography on Superose 6 increase chromatography column, connected to an FPLC AKTA from GE-Healthcare. Protein content of the column eluate was monitored online with a spectrophotometer through its optic deviation (OD) at 280 nm. As shown on FIG. 4, elution profile of 500 kDa and 50 nm permeate are very similar to that of the HPL source except the higher molecular weight proteins or protein complexes eluted before 15 min.

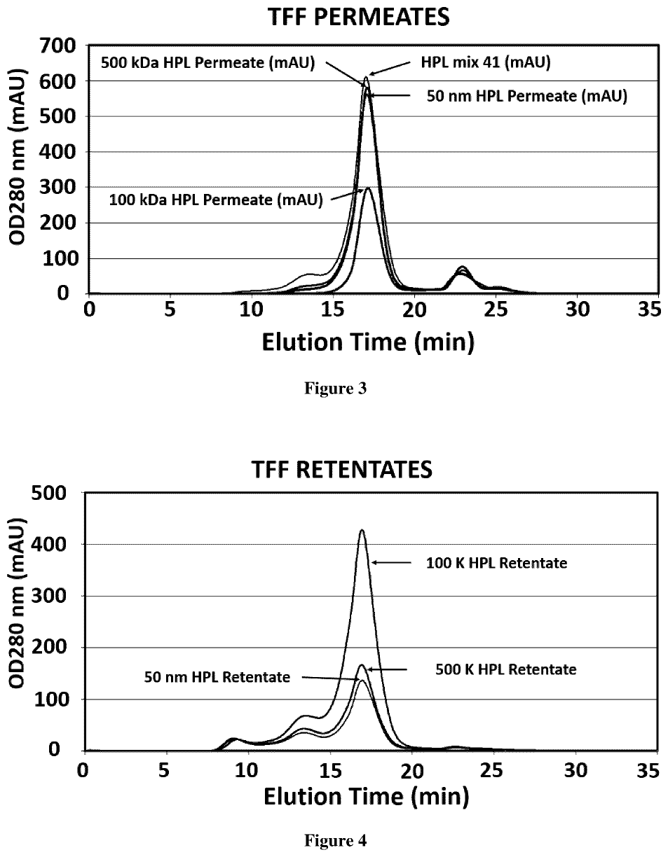

These high molecular weight components are retained in the retentate fraction (FIG. 4). The 100 kDa filter retains a great part of the protein content of the HPL since only half of the main protein, human serum albumin, was found in the permeate (FIG. 3). As a consequence, its amount in the retentate fraction was very high (FIG. 4).

The amount and size of the EV in the HPL as well as in the different permeate fractions were determined by Nanoparticle Tracking analysis (NTA) using the NS300 apparatus from MALVERN-PANALYTICAL. HPL contained a very high amount of EV, i-e 9.28 10¹⁰ EV/ml that was decreased by 96.9%, 98.6% and 98.2% using 100 kDa, 500 kDa and 50 nm filters respectively.

Taking into account all these results, the 500 kDa pore size filter was chosen for the production of EV-free HPL because it retains as much EV as the 50 nm filters with equal filtration rate and similar composition of the permeate fraction (EV-free fraction). Finally, the choice of the 500 kDa instead of the 50 nm filter has also been directed according to our hypothesis that it could produce a safer product, devoid of small size virus.

Conclusion:

Optimal conditions for EV-free HPL production were set to,
Shear force: should be between 2000 and 8000 s⁻¹ but 4000 s⁻¹ is preferred
TMP: should be between 1 and 6 psi but 2 psi is preferred.
Filter pore size should be between 100 kDa and 50 nm but 500 kDa pore size filter is preferred.

### EXAMPLE 2

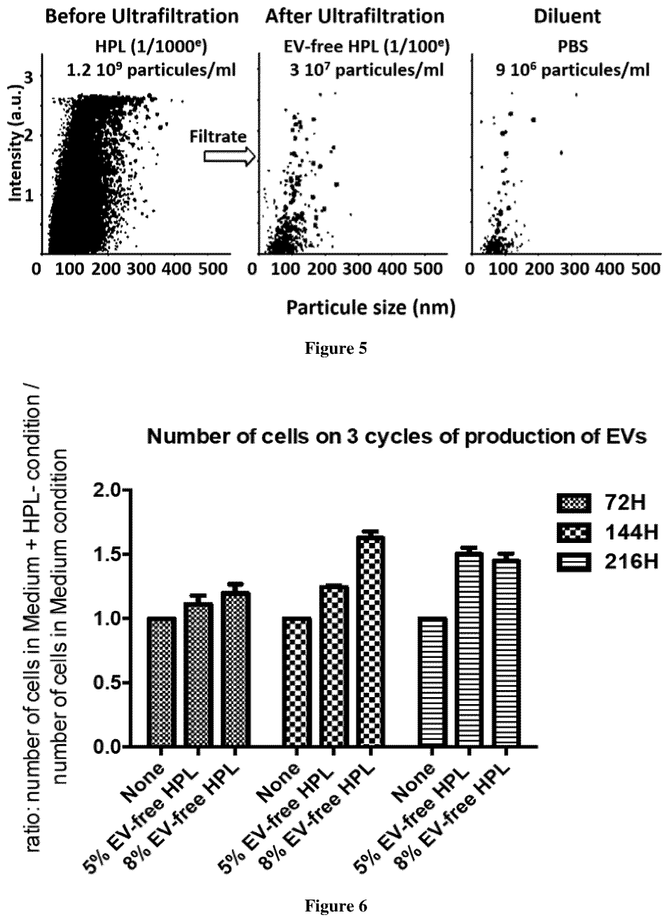

Using the parameters determined at EXAMPLE 1, another HPL batch was used to produce 1 L of EV-free HPL. Starting from a 1.1 L solution of undiluted HPL, it took about 4 h with a 155 cm² hollow-fiber filter to produce 1 L of 99.8% EV-depleted HPL as determined by NTA analysis (FIG. 5).

Increasing the filtration surface to 1600 cm² would allow the production of 10 L of EV-depleted HPL in just the same time (4h) (enough for 100 L of culture medium containing 10% of EV-depleted HPL). In this configuration, EV are retained in the retentate compartment and the permeate constitutes the EV-depleted HPL. The permeate is sterile and free of any bacteria, mycoplasma and virus. Stock of EV-free HPL can be stored frozen (−20 to −80° C.) and used in addition of any culture media, for many different cell types and at various concentration since EV-depletion of HPL occurs before dilution in the culture medium.

### EXAMPLE 3

Mesenchymal stromal cells (MSCs) are multipotent cells found in a large number of adult tissues. Many studies highlighted their ability to participate in the repair of damaged tissues. They exert immunomodulatory, anti-apoptotic, pro-angiogenic, growth support of stem or progenitor cells, anti-fibrotic or chemoattractant effects by secreting a wide range of bioactive molecules. Therefore we used bone marrow MSCs to produce EV with EV-depleted media as prepared in EXAMPLES 1 and 2.

To test the validity of using EV-free HPL-containing medium instead of basal medium without HPL or serum, we first examined the ability of this supplement to sustained MSC culture for at least 3 periods of 3 days. MSC are first amplified in their standard culture media (α-MEM supplemented with 5% HPL) and then they are “rinsed” in the presence of medium alone or medium supplemented with EV-free HPL. The cells are then placed in medium supplemented or not with EV-Free HPL for a first secretory phase of 72 h. At 72 h the culture medium is recovered for the EV quantification. A sample of cells is harvested for counting. The rest of the cells are replaced in the presence of the same culture conditions again for 72 hours. We can thus perform several cycles of production of EV.

Thus, we can show for example on human Mesenchymal Stromal Cells (MSCs), adherent stem cells derived from the human bone marrow, that the presence of EV-Free HPL allows maintaining them longer in culture compared to medium without HPL, thereby increasing the number of EV production cycles (FIG. 6).

We observe that the Medium containing EV-Free HPL (5 or 8%) preserves more cell viability than the basal Medium condition, on 3 consecutive EV production cycles. Moreover, pictures of cultured cells in the different conditions and all along the incubation time show that the presence of EV-free HPL preserves cell morphology whereas medium without HPL do not (Data not shown).

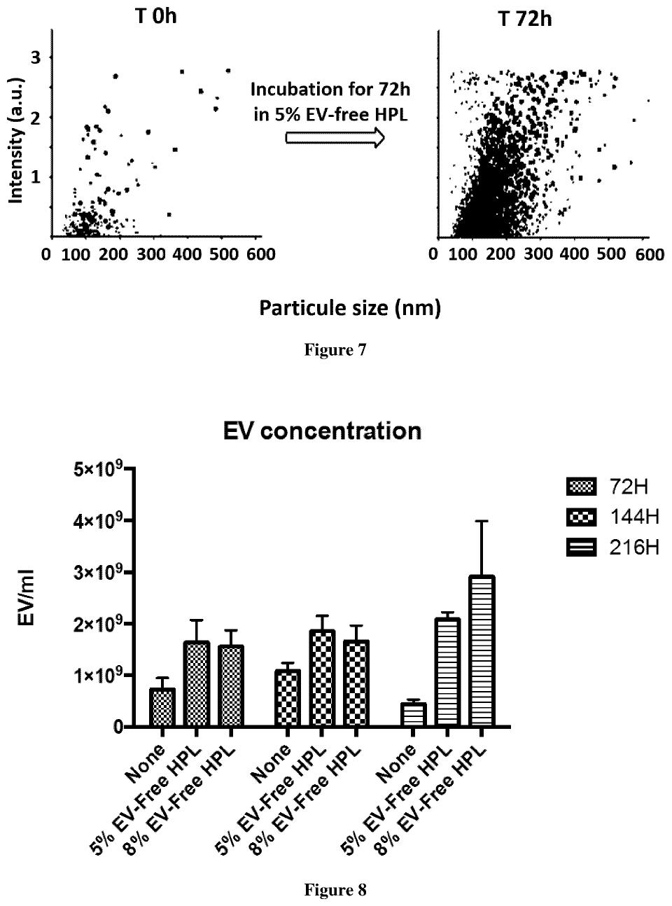

We reported on FIG. 7 the NTA quantification of EV contained in the medium either before (T 0 h) or after 72 h of MSC secretion. The results indicate that in the presence of 5% EV-free HPL, the amount of EV increases from 1.7 107/ml to 2.6 108/ml. Thus EV secreted by MSC could be calculated to represent 93.5% of total EV in this particular incubation. It means that EV secreted by MSCs represent 93.5% of the total amount of EV observed at 72 h.

NTA analyses of all the incubation media from the experiments described above are reported on FIG. 8. We observed that EV-free HPL (either 5 or 8%) allow a sustained production of EV all along the three 72 h incubation periods. EV production in medium without HPL is lower and decreases substantially during the third period. The amount of EV produced among the three consecutive cycles of secretion were respectively of 2.27 109, 5.59 109 and 6.14 109 EV/ml for HPL-free, 5% and 8% EV-free HPL containing medium. Thus, in these experiments, a single run of MSCs amplification would produce 2.4 and 2.7 more EV when incubated in the presence of respectively 5 and 8% EV-free HPL compared to HPL-free medium.

At the end of the process, EV from EV-Free HPL containing conditioned medium can be isolated and concentrated by any technical approach such as ultracentrifugation, ultrafiltration, size exclusion chromatography, precipitation (PEG or Antibodies) . . . .

Conclusion:

Therefore our preparation process of EV-Free HPL allows the production of large amounts of EV from various human cells, compatible with clinical use in different therapeutic applications. This process can be extended to any EV-containing animal media such as sera that are used to promote cell survival and/or proliferation from different animal species. It is compatible with the production of large volumes of conditioned media, including in bioreactors, allowing the large-scale production of therapeutic EV for both human and veterinary applications.

### EXAMPLE 4

Ultracentrifugation

A volume of 2 ml of fetal bovine serum (FBS) on the one hand and of human platelet Lysate (hPL) on the other hand, both pure, are centrifuged at 120,000 g for 18 hours at 22° C. (TL100 optima max XP, rotor MLS50, k factor=159). Dilution of Serum or Platelet Lysate for EV depletion of by ultracentrifugation was recommended in MISEV 2018 guidelines (J. Extracell. Vesicle. 2018, Vol 7). The effect of 1:10 dilution of both additives in αMEM culture medium on the efficiency of the UC step was also evaluated

After these ultracentrifugations, 75% of the initial volume, i.e. 1.5 ml of the supernatants is recovered. EV present before and after centrifugation (supernatants) were quantified by NTA following suitable dilutions.

Tangential Flow Filtration

TFF of both FBS and hPL were performed exactly as described in this patent. For comparison with the UC protocol, EV depletion by TFF of pure and 1:10 dilution of both additives was analyzed. EV contained FBS and haply before TFF and in the TFF filtrate fraction were quantified by NTA following suitable dilutions.

Results

The amount of EV remaining in the supernatant following UC or in the filtrate following TFF was express as a percentage of EV initially present before UC or TFF. Results are reported in the Table below as mean+/−SE of the number of determinations (n) in each condition. Pure FBS and Pure hPL refer to undiluted FBS and hPL. 10% FBS and 10% hPL refer to 1:10 dilution of FBS and hPL in αMEM medium.

| | Percentage of remaining | | Percentage of remaining | |
|---|---|---|---|---|
| | EV after UC | | EV after TFF | |
| | Mean +/− SE | n | Mean +/− SE | n |
| Pure FBS | 57.3 +/− 7.1 | 2 | 1.7 +/− 1.5 | 11 |
| αMEM 10% | 27.1 +/− 3.5 | 3 | 3.0 +/− 1.4 | 2 |
| FBS | | | | |
| Pure hPL | 71.8 +/− 9.4 | 3 | 0.6 +/− 0.6 | 5 |
| αMEM 10% | 46.5 | 1 | 0.5 | 1 |
| hPL | | | | |

Conclusion

TFF is far more efficient than UC in depleting both FBS and hPL from endogenous EV. Dilution of both FBS and hPL only marginally increase the efficiency of depletion by UC whereas depletion by TFF remains maximal.

### REFERENCES

Throughout this application, various references describe the state of the art to which this invention pertains. The disclosures of these references are hereby incorporated by reference into the present disclosure.

1. A method for preparing an extracellular vesicle (EV)-depleted medium by removing extracellular vesicles from a medium, comprising the steps of i) filtering the medium by tangential-flow filtration with a filter having a pore size between 100 kDa and 50 nm, a trans-membrane pressure (TMP) between 1 and 6 psi and a shear rate between 2000 and 8000 s⁻¹ and ii) collecting the permeate after said tangential-flow filtration, wherein the permeate is the (EV)-depleted medium.

2. The method of claim 1 wherein the medium is a serum free medium.

3. The method of claim 1 wherein the medium is a platelet lysate.

4. The method of claim 1 wherein the pore size of is about 100 kDa, about 150 kDa, about 200 kDa, about 250 kDa, about, 300 kDa, about 350 kDa, about 400 kDa, about 500 kDa, about 550 kDa, about 600 kDa, about 650 kDa, about 700, kDa, about 750 kDa, about 750 kDa, about 800 kDa, about 850 kDa, about 900 kDa, about 950 kDa, or about 1000 kDa.

5. The method of claim 1, wherein the pore size is about 500 kDa.

6. The method of claim 1 wherein the filter comprises a hollow fiber module comprising a bundle of filter membranes, each filter membrane being shaped in the form of a hollow tube.

7. The method of claim 1 wherein the TMP is about 1 psi, about 1.5 psi, about 2 psi, about 2.5 psi, about 3 psi, about 3.5 psi, about 4 psi, about 4.5 psi, about 5 psi, about 5.5 psi or about 6 psi is used.

8. The method of claim 1 wherein the TMP is about 2 psi.

9. The method of claim 1 wherein the shear rate of is about 2000 s⁻¹, about 2500 s⁻¹, about 3000 s⁻¹, about 3500 s⁻¹, about 4000 s⁻¹, about 4500 s⁻¹, about 5000 s⁻¹, about 5500 s⁻¹, about 6000 s⁻¹, about 6500 s⁻¹ about 7000 s⁻¹, about 7500 s⁻¹, or about 8000 s⁻¹.

10. The method of claim 1 wherein the shear rate is about 4000 s⁻¹.

11. An extracellular vesicles-depleted medium obtainable by the method of claim 1.

12. A method for producing extracellular vesicles from a population of cells comprising the steps of i) preparing an EV-depleted medium by the method of claim 1, ii) culturing the population of cells in a culture medium supplemented by the EV-depleted medium under conditions that allow the production of EV by the population of cells and iii) harvesting the EV that are produced at step ii).

13. The method of claim 12 wherein the population of cells is a population of mesenchymal stem cells.

14. The population of extracellular vesicles obtainable by the method of claim 12.

15. (canceled)

16. The method of claim 3 wherein the platelet lysate is a human platelet lysate.

17. A pharmaceutical composition comprising
EV prepared by the method of claim 12, wherein the EV are loaded with or coupled to a therapeutic agent; and
a pharmaceutically acceptable carrier.

18. The pharmaceutical composition of claim 17, wherein the therapeutic agent is a small molecule, a protein or a nucleic acid molecule.

19. The pharmaceutical composition of claim 17, wherein the pharmaceutical composition is formulated for topical, parenteral, intravenous, intraarterial, cutaneous, subcutaneous, intratumoral, intramuscular, intracranial, intraorbital, ophthalmic, intraventricular, intrahepatic, intracapsular, intrathecal, intracisternal, intraperitoneal, intranasal, aerosol, suppository, or oral administration.

20. A method of providing a therapeutic agent to a patient in need thereof, comprising
administering to the patient a therapeutically effective amount of the pharmaceutical composition of claim 17.
